(19) United States
(12) Patent Application Publication
(10) Pub. No.: US 2024/0130903 A1
(43) Pub. Date: Apr. 25, 2024

(54) COMPOSITE ABSORBER, AND POLYMER ABSORBENT

(71) Applicant: ORGANO CORPORATION, Tokyo (JP)

(72) Inventors: Hitoshi TAKADA, Tokyo (JP); Ryouta IWAURA, Tokyo (JP)

(73) Assignee: ORGANO CORPORATION, Tokyo (JP)

(21) Appl. No.: 18/269,649

(22) PCT Filed: Dec. 15, 2021
(86) PCT No.: PCT/JP2021/046326

§ 371 Date: Jun. 25, 2023

(30) Foreign Application Priority Data
Dec. 29, 2020 (JP) 2020-219823

(51) Int. Cl. A61F 13/53

(52) U.S. Cl. CPC A61F 13/53; A61F 2013/530226; A61F 2013/530481; A61F 2013/53089

(57) ABSTRACT

A composite absorber for absorbing liquid, the composite absorber having: a polymer absorbent having a hydrophilic continuous skeleton and continuous pores; and a highly absorbent polymer, wherein the polymer absorbent contains at least a —COOH group and —COONa group as an ion exchange group, and the total ion exchange capacity of the —COOH group and —COONa group per mass in a dry state is at least 4.0 mg equivalent/g.

### FIELD

The present invention relates to a composite absorbent body and to a polymer absorbent.

### BACKGROUND

Superabsorbent polymers (SAP) which exhibit high liquid absorption are known as absorbent bodies to be used for absorption of liquids such as aqueous solutions.

As disclosed in PTLs 1 to 5, for example, they are applied for use in a variety of fields including disposable paper diapers, civil engineering and construction materials such as condensation-proof sheets and simple soil etc., base materials for pharmaceuticals and the like, and absorbent materials for leaking liquids.

### CITATION LIST

### Patent Literature

[PTL 1] International Patent Publication No. WO2013/018571
[PTL 2] Japanese Unexamined Patent Publication No. 2017-36638
[PTL 3] Japanese Unexamined Patent Publication No. 2017-205225
[PTL 4] Japanese Unexamined Patent Publication No. 63-75016
[PTL 5] Japanese Unexamined Patent Publication No. 8-38893

### SUMMARY

### Technical Problem

Such superabsorbent polymers (SAP) are able to retain large amounts of liquid (since they have high liquid retention capacity); however, because their liquid absorption rates are slow, when used in conventional absorbent bodies they are combined with pulp so as to allow rapid temporary retention of liquids. When liquid is discharged into a conventional absorbent body, it is rapidly absorbed by the pulp in the absorbent body and temporarily retained in the pulp, and is subsequently delivered to SAP which has high liquid retention capacity, and retained in the SAP.

Liquids absorbed by absorbent bodies are generally ion-rich, and most notably the divalent ions (such as Ca²⁺ and Mg²⁺) in liquids, even when present in small amounts, can have major adverse effects on absorption performance (especially the liquid absorption, fluid retention and absorption rate) of the SAP.

However, since a conventional absorbent body is not equipped with a function of modifying the salt concentrations in liquids, the liquids that are temporarily retained in the pulp are directly delivered to SAP, which causes variation in the absorption performance of the SAP due to the effects of the liquid salt concentration (especially the divalent ion concentration), and potentially makes it impossible for the absorbent body to exhibit stable absorption performance.

The present invention has been devised in light of this problem, and its object is to provide an absorbent body that can exhibit stable absorption performance.

### Solution to Problem

One aspect of the invention (aspect 1) is a composite absorbent body for absorption of liquids, wherein:
the composite absorbent body includes a polymer absorbent comprising a hydrophilic continuous skeleton and continuous pore, and a superabsorbent polymer, and
the polymer absorbent contains at least a —COOH group and a —COONa group as ion-exchange groups, the total ion-exchange capacity for —COOH and —COONa groups being 4.0 mg equivalents/g or greater per mass in a dry state.

Since the polymer absorbent in the composite absorbent body of this aspect comprises a hydrophilic continuous skeleton and continuous pores it is able to rapidly absorb and temporarily retain liquids, and since it contains at least a specified amount of —COOH groups and —COONa groups as ion-exchange groups, the ions (especially divalent ions such as Ca²⁺ and Mg²⁺) in the liquid can undergo ion-exchange by the —COOH groups and —COONa groups when the polymer absorbent absorbs and temporarily retains liquids, thus modifying the liquid to become one that is less likely to adversely affect the absorption performance (especially the liquid absorption, fluid retention and absorption rate) of the superabsorbent polymer (SAP).

Therefore, in the composite absorbent body of this aspect, the liquid is delivered to the SAP after having been modified by the polymer absorbent, thus helping to reduce variation in the absorption performance of the SAP and enabling it to exhibit stable absorption performance as an absorbent body.

According to another aspect (aspect 2) of the invention, the polymer absorbent in the composite absorbent body of aspect 1 has an ion-exchange rate of 50% or greater for polyvalent ions.

Since the composite absorbent body of this aspect has an ion-exchange rate of 50% or greater for polyvalent ions (divalent or greater ions) in the polymer absorbent, thereby allowing the liquid to be reliably modified, it is possible to further reduce variation in the absorption performance of the SAP and to exhibit more stable absorption performance as an absorbent body.

According to another aspect (aspect 3) of the invention, the polymer absorbent in the composite absorbent body of aspect 1 or 2 has a liquid absorption of 30 g/g or greater per unit mass.

Since the composite absorbent body of this aspect has at least a fixed level of liquid absorption by the polymer absorbent and can absorb more liquid and reliably modify it, it is able to further reduce variation in the absorption performance of the SAP and can exhibit more stable absorption performance as an absorbent body.

According to another aspect (aspect 4) of the invention, the polymer absorbent in the composite absorbent body of any one of aspects 1 to 3 has a void percentage of 85% or greater per unit volume of the polymer absorbent.

Since the composite absorbent body of this aspect has at least a fixed level of void percentage by the polymer absorbent and can absorb more liquid and reliably modify it, it is able to further reduce variation in the absorption performance of the SAP and can more stably and satisfactorily exhibit absorption performance as an absorbent body.

According to another aspect (aspect 5) of the invention, the polymer absorbent in the composite absorbent body of any one of aspects 1 to 4 has a mean diameter of 1 μm to 1000 lam for the continuous pores.

Since the composite absorbent body of this aspect has a specified range for the mean diameter of the continuous pores of the polymer absorbent, the spaces (pores) of the polymer absorbent that take up liquid are unlikely to collapse, allowing a higher absorption rate to be obtained and allowing excellent stable absorption performance to be exhibited.

In particular, if the void percentage is 85% or greater per unit volume of the polymer absorbent and the mean diameter is 1 μm to 1000 lam for the continuous pores, then it will be possible to absorb and modify the liquid in more pores, which is advantageous for obtaining more excellent ion-exchange efficiency.

According to another aspect (aspect 6) of the invention, the polymer absorbent in the composite absorbent body according to any one of aspects 1 to 5 is a monolithic absorbent.

If the polymer absorbent in the composite absorbent body of this aspect is a monolithic absorbent, which allows liquid to be rapidly absorbed, it will be possible to deliver the temporarily retained liquid more reliably to the SAP, thus allowing excellent absorption performance to be more stably exhibited.

According to another aspect (aspect 7) of the invention, the polymer absorbent in the composite absorbent body according to any one of aspects 1 to 6 is a crosslinked polymer hydrolysate of a compound comprising a (meth)acrylic acid ester and two or more vinyl groups in the molecule.

If the polymer absorbent in the composite absorbent body of this aspect has this specified construction, then the hydrophilic continuous skeleton will tend to extend and the continuous pores will tend to widen during absorption of liquids, thus allowing more liquid to be more rapidly taken up into the continuous pores and providing even higher absorption performance as an absorbent body, while also allowing more liquid to be reliably modified and further reducing the possibility of variation in the absorption performance of the SAP.

According to another aspect (aspect 8) of the invention, the superabsorbent polymer in the composite absorbent body according to any one of aspects 1 to 7 is an acrylic acid-based superabsorbent polymer with a cation on the surface.

The acrylic acid-based superabsorbent polymer (SAP) with a cation on the surface is susceptible to adverse effects on absorption performance (especially liquid absorption, fluid retention and absorption rate) due to ions in liquid; however, even if the composite absorbent body of this aspect includes such a SAP, ions in the liquid undergo ion-exchange by —COOH groups and —COONa groups upon absorption and temporary retention of the liquid by the polymer absorbent, allowing the liquid to be modified, tending to reduce variation in the absorption performance of the SAP and allowing its absorption performance as an absorbent body to be stably exhibited.

One aspect (aspect 9) of the invention is a polymer absorbent to be used with a superabsorbent polymer, wherein:
the polymer absorbent comprises a hydrophilic continuous skeleton and continuous pore, and
contains at least a —COOH group and a —COONa group as ion-exchange groups, the total ion-exchange capacity for —COOH and —COONa groups being 4.0 mg equivalents/g or greater per mass in a dry state.

Since the polymer absorbent of this aspect comprises a hydrophilic continuous skeleton and continuous pores it is able to rapidly absorb and temporarily retain liquids, and since it contains at least a specified amount of —COOH groups and —COONa groups as ion-exchange groups, the ions (especially divalent ions such as Ca²⁺ and Mg²⁺) in the liquid can undergo ion-exchange by —COOH groups and —COONa groups when the polymer absorbent absorbs and temporarily retains liquids, thus modifying the liquid to become a liquid that is less likely to adversely affect the absorption performance (especially the liquid absorption, fluid retention and absorption rate) of the superabsorbent polymer (SAP).

Therefore, in the polymer absorbent according to this aspect, the liquid is transferred to the SAP after having been modified by the polymer absorbent, thus helping to reduce variation in the absorption performance of the SAP and allowing stable absorption performance to be exhibited as an absorbent body.

### Advantageous Effects of Invention

The present invention can provide an absorbent body that is able to exhibit stable absorption performance.

### BRIEF DESCRIPTION OF DRAWINGS

FIG. 1 is an exploded perspective view of a composite absorbent body 1 according to an embodiment of the invention.

FIG. 2 is an exploded perspective view of a composite absorbent body 1′ according to another embodiment of the invention.

FIG. 3 is a diagram illustrating the process of producing absorbent A as an example of a polymer absorbent.

FIG. 4 is a SEM photograph of absorbent A at 50× magnification.

FIG. 5 is a SEM photograph of absorbent A at 100× magnification.

FIG. 6 is a SEM photograph of absorbent A at 500× magnification.

FIG. 7 is a SEM photograph of absorbent A at 1000× magnification.

FIG. 8 is a SEM photograph of absorbent A at 1500× magnification.

FIG. 9 is a set of graphs showing the relationship between monovalent and divalent ion concentration in a liquid, and SAP absorption performance (liquid absorption, fluid retention and absorption rate).

FIG. 10 is a graph showing the effect of absorbent A, as an example of a polymer absorbent, on divalent ion concentration in a liquid.

### DESCRIPTION OF EMBODIMENTS

A preferred embodiment of the invention will now be described in detail using the composite absorbent body 1 as a working embodiment.

Throughout the present specification, unless otherwise specified, the concept of “viewing an object (for example, a composite absorbent body) on the horizontal plane in the expanded state in the thickness direction of the object, from the upper side in the vertical direction”, will be referred to by the phrase “as viewed flat”.

### [Composite Absorbent Body]

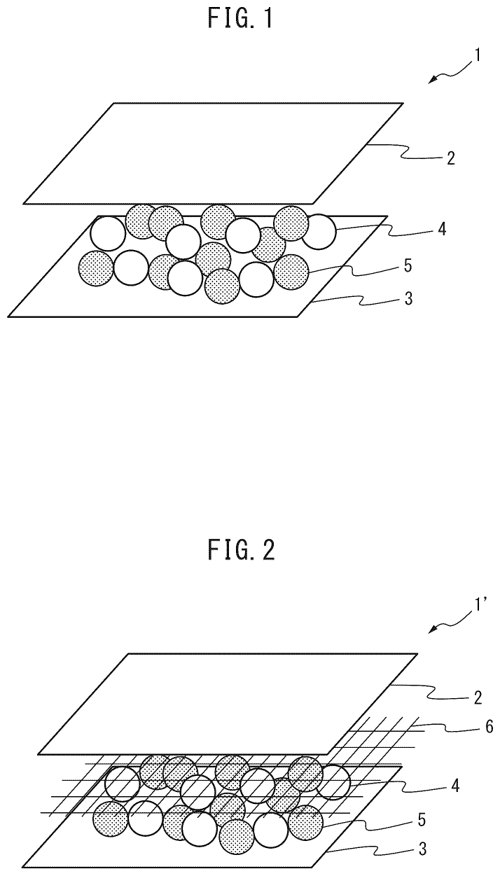

FIG. 1 is an exploded perspective view of a composite absorbent body 1 according to one embodiment of the invention.

The composite absorbent body 1 shown in FIG. 1 comprises, as the basic construction, a first retaining sheet 2 having an essentially rectangular outer shape as viewed flat and forming the surface on one side of the composite absorbent body 1, in the thickness direction, a second retaining sheet 3 forming the surface on the other side of the composite absorbent body 1, and a liquid absorbing member situated between the two aforementioned sheets and comprising a mixture of a polymer absorbent 4 and a superabsorbent polymer 5 (SAP).

The liquid absorbing member in the composite absorbent body 1 is situated between the first retaining sheet 2 and second retaining sheet 3 and is constructed with a polymer absorbent 4 having a hydrophilic continuous skeleton and continuous pores, and a superabsorbent polymer 5, allowing it to absorb and retain liquid that has permeated through the first retaining sheet 2.

The polymer absorbent also contains at least a —COOH group and a —COONa group as ion-exchange groups and has characteristic ion-exchange ability such that the total ion-exchange capacity for —COOH and —COONa groups is 4.0 mg equivalents/g or greater per mass in a dry state.

Since the polymer absorbent in the composite absorbent body 4 comprises a hydrophilic continuous skeleton and continuous pores, it is able to rapidly absorb and temporarily retain liquids, and since it contains at least a specified amount of —COOH groups and —COONa groups as ion-exchange groups, the ions (especially divalent ions such as Ca²⁺ and Mg²⁺) in the liquid can undergo ion-exchange by —COOH groups and —COONa groups when the polymer absorbent absorbs and temporarily retains liquids, thus modifying the liquid to become a liquid that is less likely to adversely affect the absorption performance (especially the liquid absorption, fluid retention and absorption rate) of the SAP.

The composite absorbent body 4 can therefore deliver liquid to the SAP after having been modified by the polymer absorbent, thus helping to reduce variation in the absorption performance of the SAP and allowing stable absorption performance to be exhibited as an absorbent body.

According to the invention, the liquid absorbing member is not limited to the aspects of the composite absorbent body 1 of this embodiment, and the liquid absorbing member may also include other liquid-absorbing materials so long as it includes the polymer absorbent and SAP exhibiting at least the characteristic liquid absorption behavior described above.

The structure of the composite absorbent body of the invention is also not limited to the aspects of the composite absorbent body 1 of this embodiment, and the composite absorbent body may, for example, also have a hydrophilic fiber sheet 6 situated between the first retaining sheet 2 and the liquid absorbing member (i.e., the polymer absorbent 4 and superabsorbent polymer 5), as in the composite absorbent body 1′ of the other embodiment of the invention shown in FIG. 2.

According to the invention, the outer shape and various dimensions and basis weight of the composite absorbent body are not particularly restricted, so long as the effect of the invention is not inhibited, and any desired outer shape (such as circular, oblong, polygonal, hourglass or design shape), and dimensions and basis weight, may be employed, depending on the intended purpose and manner of use.

Each of the structural members of the composite absorbent body of the invention will now be explained in further detail using the composite absorbent body 1 of the embodiment shown in FIG. 1 as an example.

### (Retaining Sheet)

In the composite absorbent body 1 shown in FIG. 1, the first retaining sheet 2 forming the surface on one side of the composite absorbent body 1 has an essentially rectangular outer shape similar to the outer shape of the composite absorbent body 1, as viewed flat. The first retaining sheet 2 may be formed of a liquid-permeable sheet-like member that allows permeation of liquid supplied to the composite absorbent body 1 and causes it to be absorbed and retained in the inner liquid absorbing member.

The first retaining sheet 2 has a slightly larger size overall compared to the liquid absorbing member situated on the inner side (i.e., compared to the regions where the liquid-absorbing materials such as the polymer absorbent 4 are situated), while at the perimeter edges it is bonded with the second retaining sheet 3 situated on the other side in the thickness direction of the composite absorbent body 1, using any adhesive or heat sealing means.

The second retaining sheet 3 forming the surface on the other side of the composite absorbent body 1 likewise has an essentially rectangular outer shape similar to the outer shape of the composite absorbent body 1, as viewed flat. The second retaining sheet 3 is formed of a liquid-impermeable sheet-like member that prevents liquid that was not absorbed and retained by the inner liquid absorbing member and liquid that has seeped from the liquid absorbing member, from leaking out of the composite absorbent body 1.

According to the invention, the respective sheet-like members to be used for the first retaining sheet and second retaining sheet are not limited to this embodiment, and the composite absorbent body of the invention may have the retaining sheets of either or both the first retaining sheet and second retaining sheet formed of liquid-permeable sheet-like members. In other words, the composite absorbent body of the invention may have the retaining sheet of either or both the first retaining sheet and second retaining sheet formed of a liquid-impermeable sheet-like member.

When a liquid-permeable sheet-like member is used as a retaining sheet, the liquid-permeable sheet-like member is not particularly restricted, so long as it does not interfere with the effect of the invention, and any liquid-permeable sheet-like member may be employed, depending on the purpose and intended usage. Examples of liquid-permeable sheet-like members include nonwoven fabrics such as hydrophilic air-through nonwoven fabrics, spunbonded nonwoven fabrics and point-bonded nonwoven fabrics, or woven fabrics, knitted fabrics and porous resin films.

When a hydrophilic nonwoven fabric, woven fabric or knitted fabric (hereunder collectively referred to as “fiber sheet”) is used as the liquid-permeable sheet-like member, the fiber sheet may have a monolayer structure or a multilayer structure with two or more layers. The type of constituent fibers of the fiber sheet is not particularly restricted, and examples include hydrophilic fibers such as cellulosic fibers or hydrophilicized thermoplastic resin fibers. Such fibers may be used alone, or two or more different types of fibers may be used in combination.

Examples of cellulosic fibers to be used as constituent fibers for the fiber sheet include natural cellulose fibers (such as cotton or other plant fibers), regenerated cellulose fibers, refined cellulose fibers and semi-synthetic cellulose fibers. Examples of thermoplastic resin fibers to be used as constituent fibers for the fiber sheet include fibers made of publicly known thermoplastic resins, including olefin-based resins such as polyethylene (PE) and polypropylene (PP), polyester-based resins such as polyethylene terephthalate (PET), and polyamide-based resins such as 6-nylon. Such resins may be used alone, or two or more resins may be used in combination.

When a liquid-impermeable sheet-like member is used as a retaining sheet, the liquid-impermeable sheet-like member is not particularly restricted, so long as it does not interfere with the effect of the invention, and any liquid-impermeable sheet-like member may be employed, depending on the purpose and intended usage. Examples of such liquid-impermeable sheet-like members include hydrophobic nonwoven fabrics formed of any desired hydrophobic thermoplastic resin fibers (for example, polyolefin-based fibers such as PE and PP, polyester-based fibers such as PET, and various composite fibers such as core-sheath fibers); porous or non-porous resin films formed of hydrophobic thermoplastic resins such as PE or PP; laminates of nonwoven fabrics attached to resin films; and layered nonwoven fabrics such as SMS nonwoven fabrics.

According to the invention, the outer shape and various dimensions and basis weight of the retaining sheet are not particularly restricted, so long as the effect of the invention is not inhibited, and any desired outer shape (such as circular, oblong, polygonal, hourglass or design shape), and dimensions and basis weight, may be employed, depending on the intended purpose and manner of use.

### (Liquid Absorbing Member)

The liquid absorbing member in the composite absorbent body 1 shown in FIG. 1 is situated between the first retaining sheet 2 and second retaining sheet 3 and is constructed with a polymer absorbent 4 having a hydrophilic continuous skeleton and continuous pores, and a superabsorbent polymer 5, allowing it to absorb and retain liquid that has permeated through the first retaining sheet 2, as mentioned above.

The polymer absorbent 4 and superabsorbent polymer 5 of the liquid absorbing member in the composite absorbent body 1 are bonded with the first retaining sheet 2 and second retaining sheet 3 by an adhesive such as a hot-melt adhesive; however, the polymer absorbent does not need to be bonded with the retaining sheet in the composite absorbent body of the invention.

As mentioned above, the liquid absorbing member of the invention includes, as essential constituent components, a polymer absorbent with characteristic ion-exchange capacity comprising a hydrophilic continuous skeleton and continuous pores, and a superabsorbent polymer. The polymer absorbent is described in detail below; however, the superabsorbent polymer essentially consists of powder or granules composed of a SAP (Super Absorbent Polymer) known in the field, such as sodium acrylate copolymer.

The specific type of superabsorbent polymer (SAP) is not particularly restricted, and for example, an acrylic acid-based SAP having cations present on the surface is suitable for use. The acrylic acid-based SAP with a cation on the surface is susceptible to adverse effects on absorption performance (especially liquid absorption, fluid retention and absorption rate) due to ions in liquid; however, even if the composite absorbent body 4 includes such a SAP, ions in the liquid undergo ion-exchange by —COOH groups and —COONa groups upon absorption and temporary retention of the liquid by the polymer absorbent, allowing the liquid to be modified, which tends to reduce variation in the absorption performance of the SAP and allows it to exhibit stable absorption performance as an absorbent body.

According to the invention, the liquid absorbing member situated between the first retaining sheet and second retaining sheet may include the polymer absorbent and SAP alone as liquid-absorbing materials, or it may also additionally include a liquid-absorbing material that is publicly known in the field. Examples of such liquid-absorbing materials include hydrophilic fibers, and more specifically cellulose-based fibers, such as pulp fibers (ground pulp, for example), cotton, rayon or acetate.

According to the invention, the outer shape (the planar shape in the region where the liquid-absorbing material is present) and various dimensions and basis weight of the liquid absorbing member are not particularly restricted, so long as the effect of the invention is not inhibited, and any desired outer shape or dimensions and basis weight may be employed, depending on the desired liquid absorption, flexibility and strength.

### (Hydrophilic Fiber Sheet)

The composite absorbent body of the invention may also have a hydrophilic fiber sheet 6 between the first retaining sheet 2 and the liquid absorbing member (i.e., the polymer absorbent 4 and superabsorbent polymer 5), as in the composite absorbent body 1′ of another embodiment shown in FIG. 2.

According to the invention, the hydrophilic fiber sheet used in the composite absorbent body is not particularly restricted, so long as it does not interfere with the effect of the invention, and any hydrophilic fiber sheet may be employed according to the intended purpose and usage. Examples of such hydrophilic fiber sheets include hydrophilic nonwoven fabrics, woven fabrics and knitted fabrics. The hydrophilic fiber sheet may have a monolayer structure, or may have a multilayer structure with two or more layers.

The type of constituent fibers of the hydrophilic fiber sheet is not particularly restricted, and examples include hydrophilic fibers such as cellulosic fibers or hydrophilicized thermoplastic resin fibers. Such fibers may be used alone, or two or more different types of fibers may be used in combination.

Examples of cellulosic fibers to be used as constituent fibers for the hydrophilic fiber sheet include natural cellulose fibers (such as cotton or other plant fibers), regenerated cellulose fibers, refined cellulose fibers and semi-synthetic cellulose fibers. Examples of thermoplastic resin fibers to be used as constituent fibers for the hydrophilic fiber sheet include fibers made of publicly known thermoplastic resins, including olefin-based resins such as PE and PP, polyester-based resins such as PET, and polyamide-based resins such as 6-nylon. Such resins may be used alone, or two or more resins may be used in combination.

According to the invention, the outer shape and various dimensions and basis weight of the hydrophilic fiber sheet are not particularly restricted, so long as the effect of the invention is not inhibited, and any desired outer shape or dimensions and basis weight may be employed, depending on the intended purpose and usage.

The polymer absorbent to be used in the composite absorbent body of the invention will now be explained in further detail.

### [Polymer Absorbent]

The polymer absorbent of the invention is not particularly restricted so long as it comprises a hydrophilic continuous skeleton and continuous pores, contains at least a —COOH group and a —COONa group as ion-exchange groups, and has characteristic ion-exchange ability such that the total ion-exchange capacity for —COOH and —COONa groups is 4.0 mg equivalents/g or greater per mass in a dry state. Examples of such polymer absorbents include polymer compounds that are hydrolysates of crosslinked polymers of two or more monomers including at least a (meth)acrylic acid ester, and that have at least one hydrophilic group as a functional group. More specifically, such examples are polymer compounds that are hydrolysates of crosslinked polymers of a (meth)acrylic acid ester and a compound with two or more vinyl groups in the molecule, and that have at least a —COOH group and a —COONa group. Such polymer absorbents are organic porous bodies having at least one —COONa group in the molecule, and also having a —COOH group. The —COONa groups are essentially evenly distributed throughout the skeleton of the porous body.

If the polymer absorbent is a hydrolysate of a crosslinked polymer of a (meth)acrylic acid ester and a compound with two or more vinyl groups in the molecule, then the hydrophilic continuous skeleton will tend to stretch (i.e. expand) upon absorption of liquid such as aqueous solutions, and the continuous pores will also tend to widen, allowing more liquid to be more rapidly taken up into the continuous pores. As a result, a composite absorbent body including such a polymer absorbent can exhibit even higher absorption performance as an absorbent body while also reliably modifying more liquid, and further reducing variation in the absorption performance of the SAP.

As used herein, “(meth)acrylic acid ester” refers to an acrylic acid ester or methacrylic acid ester.

In a polymer absorbent formed by a hydrolysate of a crosslinked polymer comprising a (meth)acrylic acid ester and divinylbenzene, the hydrophilic continuous skeleton is formed of an organic polymer having at least a —COONa and a —COOH group, and between the skeleton it has communicating pores (continuous pores) serving as sites for absorption of liquid.

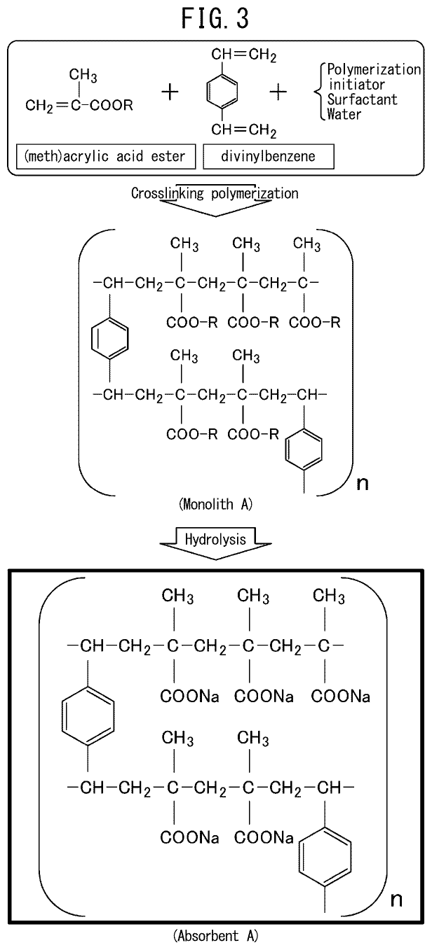

Since hydrolysis converts the —COOR group (carboxylic acid ester group) of the crosslinked polymer to a —COONa or —COOH group (see FIG. 3), the polymer absorbent may have a —COOR group.

The presence of —COOH groups and —COONa groups in the organic polymer forming the hydrophilic continuous skeleton and the total ion-exchange capacity for —COOH and —COONa groups per mass in a dry state can be confirmed by infrared spectrophotometry and quantitative analysis of the weakly acidic ion-exchange groups.

FIG. 3 is a diagram illustrating the process of producing absorbent A as an example of a polymer absorbent. FIG. 3 shows the constituent starting materials for polymerization at top, monolith A as a crosslinked polymer of a (meth)acrylic acid ester and divinylbenzene, at center, and absorbent A obtained by hydrolysis and drying treatment of monolith A, at bottom.

The following explanation concerns absorbent A, formed by hydrolysis of a crosslinked polymer of a (meth)acrylic acid ester and divinylbenzene, as an example of a polymer absorbent.

The polymer absorbent is not limited to absorbent A, and may be a hydrolysate of a crosslinked polymer of a (meth)acrylic acid ester and a compound having two or more vinyl groups in the molecule, or a hydrolysate of a crosslinked polymer of two or more monomers including at least a (meth)acrylic acid ester.

However, if the polymer absorbent is a monolithic absorbent, liquid can be rapidly absorbed and it is possible to more reliably deliver the liquid temporarily retained in the polymer absorbent to the SAP, thus allowing a composite absorbent body containing the polymer absorbent to more stably exhibit excellent absorption performance.

In the following explanation, “monolith A” refers to a “monolithic organic porous body”, which is an organic porous body comprising a crosslinked polymer of a (meth)acrylic acid ester and divinylbenzene before hydrolysis.

The term “absorbent A” is a hydrolysate of the crosslinked polymer (monolith A) of the (meth)acrylic acid ester and divinylbenzene after hydrolysis and drying treatment. In the following explanation, “absorbent A” refers to the dry form.

The structure of absorbent A will be described first.

As explained above, absorbent A has a hydrophilic continuous skeleton and continuous pores. As shown in FIG. 3, absorbent A which is an organic polymer having a hydrophilic continuous skeleton, can be obtained by crosslinking polymerization of a (meth)acrylic acid ester as the polymerization monomer, and divinylbenzene as the crosslinking monomer, and hydrolysis of the obtained crosslinked polymer (monolith A).

The organic polymer forming the hydrophilic continuous skeleton has, as structural units, an ethylene group polymer residue (hereunder, “structural unit X”) and a divinylbenzene crosslinking polymer residue (hereunder, “structural unit Y”).

The ethylene group polymer residue (structural unit X) in the organic polymer forming the hydrophilic continuous skeleton has both —COOH and —COONa groups generated by hydrolysis of carboxylic acid ester groups. When the polymerization monomer is a (meth)acrylic acid ester, the polymer residue (structural unit X) of the ethylene group has a —COONa, —COOH group and ester group.

In the absorbent A, the proportion of divinylbenzene crosslinking polymer residue (structural unit Y) in the organic polymer forming the hydrophilic continuous skeleton may be 0.1 to 30 mol %, and preferably 0.1 to 20 mol %, with respect to the total structural units. For example, in absorbent A where butyl methacrylate is the polymerization monomer and divinylbenzene is the crosslinking monomer, the proportion of divinylbenzene crosslinking polymer residue (structural unit Y) in the organic polymer forming the hydrophilic continuous skeleton may be about 3%, for example, and is preferably 0.1 to 10 mol %, and more preferably 0.3 to 8 mol %, with respect to the total structural units.

If the proportion of divinylbenzene crosslinking polymer residue in the organic polymer forming the hydrophilic continuous skeleton is 0.1 mol % or greater, the strength of the absorbent A will be unlikely to decrease, and if the proportion of divinylbenzene crosslinking polymer residue is 30 mol % or lower, absorption of liquids to be absorbed will be unlikely to decrease.

The organic polymer forming the hydrophilic continuous skeleton in the absorbent A may be composed entirely of structural unit X and structural unit Y, or may have a structural unit other than structural unit X, and structural unit Y, i.e. a polymer residue of monomers other than (meth)acrylic acid ester and divinylbenzene, in addition to structural unit X and structural unit Y.

Examples of structural units other than structural unit X and structural unit Y include polymer residues of monomers such as styrene, α-methylstyrene, vinyltoluene, vinylbenzyl chloride, glycidyl (meth)acrylate, isobutene, butadiene, isoprene, chloroprene, vinyl chloride, vinyl bromide, vinylidene chloride, tetrafluoroethylene, (meth)acrylonitrile, vinyl acetate, ethylene glycol di(meth)acrylate, tripropyleneglycol di(meth)acrylate and trimethylolpropane tri(meth)acrylate.

The proportion of structural units other than structural unit X and structural unit Y in the organic polymer forming the hydrophilic continuous skeleton may be 0 to 50 mol %, and preferably 0 to 30 mol %, with respect to the total structural units.

The absorbent A preferably has a hydrophilic continuous skeleton thickness of 0.1 to 100 μm. If the thickness of the hydrophilic continuous skeleton of absorbent A is 0.1 μm or greater, the spaces (pores) for uptake of liquid in the porous body will be unlikely to collapse during absorption, so that liquid absorption will be unlikely to decrease. A hydrophilic continuous skeleton thickness of 100 μm or lower will tend to result in an excellent absorption rate.

Since the pore structure of the hydrophilic continuous skeleton of absorbent A is an open-cell structure, the thickness of the continuous skeleton is evaluated by measuring the skeleton cross-section appearing in an electron microscope test piece. Since the continuous skeleton is formed at gaps where water (in the form of droplets) has been removed by dehydrating/drying treatment after hydrolysis, it usually has a polygonal shape. The thickness of the continuous skeleton is therefore the average of the diameters (μm) of circumscribed circles in the polygonal cross-section. In rare cases the polygonal shapes will contain small open holes, and in such cases, circumscribed circles surrounding the small holes are measured in the polygonal cross-sections.

The absorbent A also preferably has continuous pores with a mean diameter of 1 μm to 1000 μm. If the mean diameter of the continuous pores of absorbent A is 1 μm or greater, the spaces (pores) for uptake of liquid in the porous body will be unlikely to collapse during absorption, so that the absorption rate will be unlikely to decrease. A mean continuous pore diameter of 1000 μm or lower will tend to result in an excellent absorption rate. A composite absorbent body comprising the absorbent A can therefore stably exhibit excellent absorption performance.

In particular, if the void percentage is 85% or greater per unit volume of the polymer absorbent and the mean diameter is 1 μm to 1000 μm for the continuous pores, then it will be possible to absorb and modify the liquid in more pores, which is advantageous for obtaining more excellent ion-exchange efficiency.

The mean diameter (μm) of the continuous pores of the absorbent A can be measured by the mercury intrusion method, using the maximum of the pore distribution curve obtained by the mercury intrusion method. Each sample for measurement of the continuous pore mean diameter is used after drying for 18 hours or longer in a vacuum dryer set to a temperature of 50° C., regardless of the ionic form of the absorbent A. The final ultimate pressure is 0 Torr.

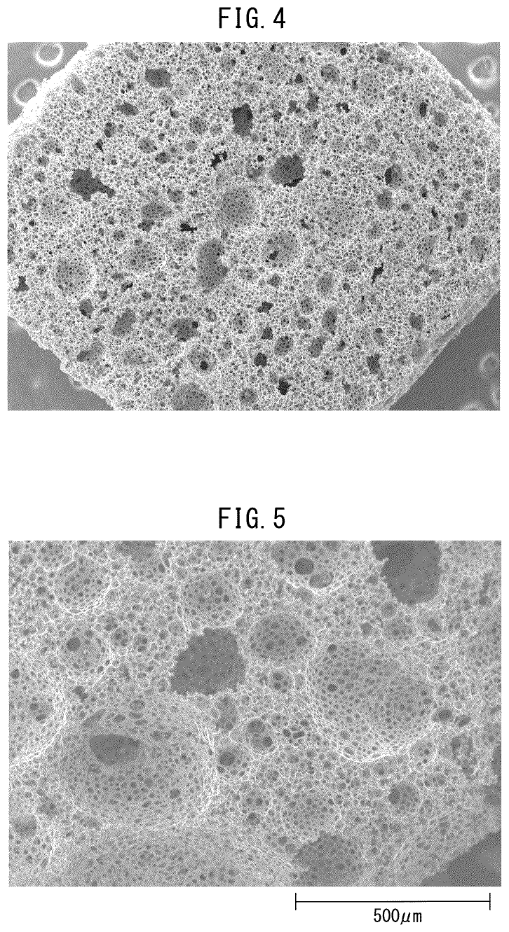

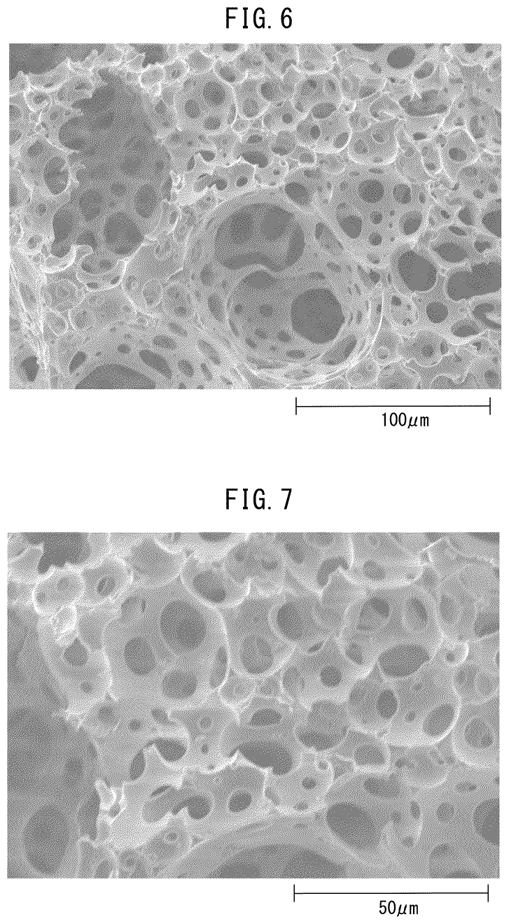

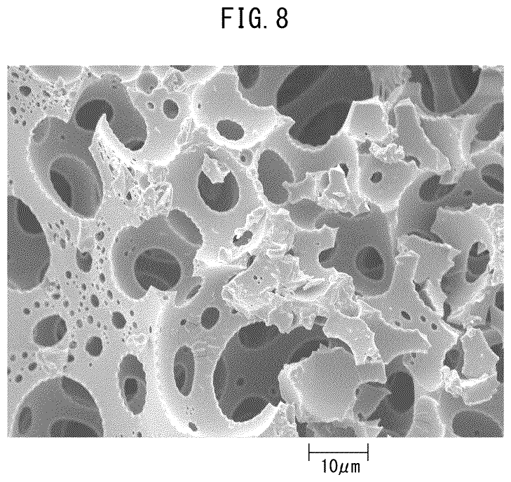

FIG. 4 is a SEM photograph of absorbent A at 50× magnification, FIG. 5 is a SEM photograph of absorbent A at 100× magnification, FIG. 6 is a SEM photograph of absorbent A at 500× magnification, FIG. 7 is a SEM photograph of absorbent A at 1000× magnification, and FIG. 8 is a SEM photograph of absorbent A at 1500× magnification.

Absorbent A shown in FIG. 4 to FIG. 8 is an absorbent having butyl methacrylate as the polymerization monomer and divinylbenzene as the crosslinking monomer, each with a 2 mm-square cuboid structure.

Absorbent A shown in FIG. 4 to FIG. 8 also has numerous foamed macropores, with overlapping sections between the foamed macropores. Absorbent A has an open-cell construction with openings (mesopores) connecting the overlapping sections between the macropores, or in other words, it is an “open-cell structure” (continuous macropore structure).

The overlapping sections between the macropores are connected openings (mesopores) with a dry mean diameter of 1 to 1000 μm, preferably 10 to 200 μm and most preferably 20 to 100 lam, the majority of which form an open pore structure. A dry mean diameter of 1 μm or greater for the mesopores will result in a more satisfactory absorption rate for liquid to be absorbed. A dry mean diameter of 1000 lam or smaller for the mesopores will tend to avoid brittleness of the absorbent A.

The number of overlapping macropores are about 1 to 12 for each macropore, and about 3 to 10 for most.

If the absorbent A has such an open-cell structure it will be possible to form homogeneous macropore groups and mesopore groups, while also significantly increasing the pore volume and area-to-weight ratio, compared to the particle-aggregate porous body described in Japanese Unexamined Patent Publication HEI No. 8-252579.

The total pore volume of pores (holes) in the absorbent A is preferably 0.5 to 50 mL/g and more preferably 2 to 30 mL/g. If the total pore volume of the absorbent A is 0.5 mL/g or greater, the spaces (holes) for uptake of liquid in the porous body will be unlikely to collapse during absorption, so that the liquid absorption and absorption rate will be unlikely to decrease. If, on the other hand, the total pore volume of the absorbent A is 50 mL/g or lower, the strength of the absorbent A will be unlikely to decrease.

The total pore volume can be measured by the mercury intrusion method. The measuring sample for total pore volume is used after drying for 18 hours or longer in a vacuum dryer set to a temperature of 50° C., regardless of the ionic form of the absorbent A. The final ultimate pressure is 0 Torr.

The state where the absorbent A has contacted liquid will be described below, the description also applying to contact between liquid and a liquid absorbing member or composite absorbent body that includes absorbent A.

The continuous pores of the absorbent A shown in FIG. 4 to FIG. 8 are holes mutually connecting multiple pores (holes), the numerous holes being outwardly visible to the naked eye. When liquid contacts with an absorbent A comprising such numerous holes, the hydrophilic continuous skeleton immediately takes up a portion of the liquid by osmotic pressure, thereby stretching (i.e. expanding). Stretching of the continuous skeleton takes place in essentially all directions. As the outer shape of the absorbent A enlarges due to stretching of the continuous skeleton during liquid absorption, the sizes of each of the holes in the absorbent A also increase. The increasing sizes of the holes increase the inner volume of the holes, thereby also increasing the amount of liquid that can be retained in the holes. The absorbent A that has thus enlarged by absorption of a certain amount of liquid is then able to absorb an additional amount of liquid in the enlarged holes, by capillary movement.

Since the liquid that has been absorbed in the hydrophilic continuous skeleton of absorbent A is less easily released from the continuous skeleton while the liquid that has been absorbed in the continuous pores is more readily released, the liquid absorbed in the continuous pores inside the composite absorbent body is released and delivered to the highly liquid retaining SAP, thus being firmly retained in the SAP.

Of the liquid absorbed into absorbent A, the amount of liquid retained in the pores is greater than the amount of liquid absorbed into the hydrophilic continuous skeleton. Since most of the liquid absorption by absorbent A takes place by retaining liquid in the pores by capillary movement, a larger void percentage, as the proportion of volume of pore voids (total pore volume) (in other words, the volume of pore voids per unit volume of the absorbent A), allows greater absorption of liquid.

The void percentage per unit volume of the polymer absorbent is preferably 85% or greater and more preferably 90% or greater. If the void percentage per unit volume of the polymer absorbent is 85% or greater it will be possible to reliably absorb and modify more liquid, helping to reduce variation in the absorption performance of the SAP. This will allow the composite absorbent body containing the polymer absorbent to more stably and satisfactorily exhibit absorption performance as an absorbent body.

For example, the void percentage of the absorbent A in FIG. 4 to FIG. 8 was determined as follows.

First, the area-to-weight ratio of absorbent A obtained by the mercury intrusion method was 400 m²/g, and the pore volume was 15.5 mL/g. A pore volume of 15.5 mL/g means that the volume of pores in 1 g of absorbent A is 15.5 mL.

Assuming a specific gravity of 1 g/mL for the absorbent A, the volume occupied by pores in 1 g of absorbent A (the pore volume) is 15.5 mL, the volume of 1 g of absorbent A being 1 mL.

Thus, the total volume of 1 g of absorbent A is 15.5+1 (mL), the pore volume ratio of which is the void percentage, and therefore the void percentage of absorbent A is 15.5/(15.5+1)×100≈94%.

According to the invention, absorbent A (the polymer absorbent) comprising the hydrophilic continuous skeleton and continuous pores is suitable for application in a composite absorbent body in the form of particulates or a sheet, for example.

Since the polymer absorbent comprises at least a —COOH group and a —COONa group as ion-exchange groups, and has a characteristic ion-exchange capacity, i.e. a total ion-exchange capacity for —COOH and —COONa groups of 4.0 mg equivalents/g or greater per mass in a dry state as mentioned above, then when the polymer absorbent absorbs and temporarily retains liquids, the ions (especially divalent ions such as Ca²⁺ and Mg²⁺) in the liquid can undergo ion-exchange by —COOH groups and —COONa groups, thus modifying the liquid to become one that is less likely to adversely affect the absorption performance (especially the liquid absorption, fluid retention and absorption rate) of the superabsorbent polymer (SAP). A composite absorbent body using the polymer absorbent can therefore deliver liquid to the SAP after having been modified by the polymer absorbent, thus helping to reduce variation in the absorption performance of the SAP and allowing stable absorption performance to be exhibited as an absorbent body.

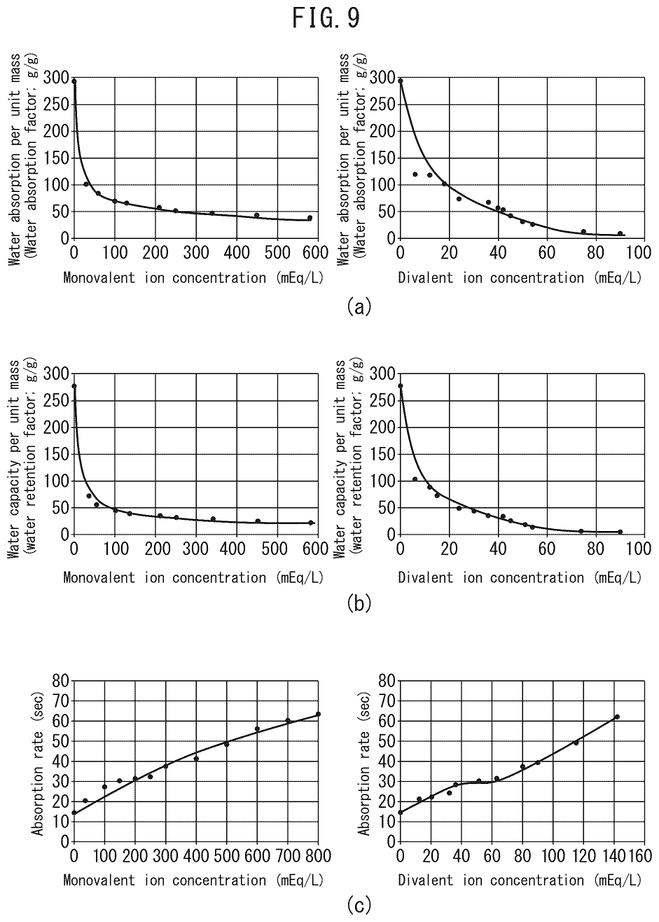

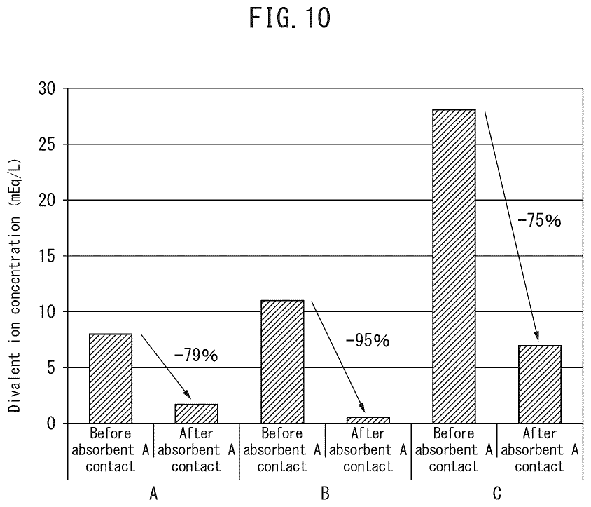

FIG. 9 is a set of graphs showing the relationship between monovalent and divalent ion concentration in a liquid, and SAP absorption performance (liquid absorption, fluid retention and absorption rate), and FIG. 10 is a graph showing the effect of absorbent A, as an example of a polymer absorbent, on divalent ion concentration in a liquid.

As shown in FIG. 9, the liquid absorption, fluid retention and absorption rate of the SAP decreased as the monovalent and divalent ion concentration of the liquid increased. In particular, it is seen that the divalent ion concentration has a major effect on the SAP absorption performance, with the liquid absorption, fluid retention and absorption rate of the SAP being significantly reduced even at very low concentrations.

Thus, the ion concentration in the liquid has a major adverse effect on SAP absorption performance, and since the ion concentration varies for different liquids, the SAP absorption performance likewise varies correspondingly.

However, since the absorbent A as an example of the invention comprises at least a —COOH group and a —COONa group as ion-exchange groups, and has a characteristic ion-exchange capacity, i.e. a total ion-exchange capacity for —COOH and —COONa groups of 4.0 mg equivalents/g or greater per mass in a dry state, the ions (especially divalent ions) in the liquid can undergo ion-exchange by the —COOH groups and —COONa groups, as shown in FIG. 10, allowing the ion concentration in the liquid to be greatly reduced, i.e. modifying the liquid so that it is less likely to adversely affect the absorption performance of the SAP.

The graph in FIG. 10 shows rates of change in ion concentration before and after contact between actual urine from 3 different humans and with different divalent ion concentrations (urine A, B and C), and absorbent A (in other words, the ion-exchange rate of absorbent A), as measured in the following manner.

First, the divalent ion concentrations (mEq/L) of each of the three urines A, B and C are measured using an ion meter (HORIBA LAQUAtwin-Ca-11 compact calcium ion meter by Horiba Advanced Techno Co., Ltd.). The measured ion concentrations are recorded as the ion concentrations “before absorbent A contact”.

Next, 0.2 g of polymer absorbent (absorbent A) is placed in a glass filter (climbing glass filter, Model: 0777-01-101, outer diameter×foot length (mm):φ7×80, filter diameter: φ20 mm, volume: 30 mL, material: borosilicate glass, pore size: 100 to 120 μm), and 30 mL of urine is poured in, using the ion meter to measure the divalent ion concentration (mEq/L) of the resulting filtrate. The measured ion concentrations were recorded as the ion concentrations “after absorbent A contact”.

The ion concentration after absorbent A contact was subtracted from the ion concentration before absorbent A contact to calculate the change in ion concentration (mEq/L) before and after absorbent A contact, and the change in ion concentration (mEq/L) before and after absorbent A contact was also divided by the ion concentration before absorbent A contact and multiplied by 100 to calculate the rate of change (%) in the divalent ion concentration for urines A, B and C.

The measurement was carried out under conditions with a temperature of 25° C. and a humidity of 60%.

Absorbent A as an example of the invention has characteristic ion-exchange capacity not found in liquid-absorbing materials of the prior art, allowing it to rapidly absorb and temporarily retain liquids, while also carrying out ion-exchange of ions (especially divalent ions) in the liquid to allow modification of the liquid to a liquid that is less likely to adversely affect the absorption performance of the SAP.

A composite absorbent body comprising absorbent A (polymer absorbent) can thus deliver liquid to the SAP after having been modified by the polymer absorbent, thus helping to reduce variation in the absorption performance of the SAP and allowing stable absorption performance to be exhibited as an absorbent body.

According to the invention, the total ion-exchange capacity of the polymer absorbent for —COOH and —COONa groups per mass in a dry state is preferably 6.0 mg equivalents/g or greater and more preferably 8.0 mg equivalents/g or greater.

According to the invention, the polymer absorbent preferably has an ion-exchange rate of 50% or greater for polyvalent ions (i.e., divalent or greater ions). If the ion-exchange rate of the polymer absorbent for polyvalent ions is 50% or greater it will be possible to more reliably modify the liquid, helping to reduce variation in the absorption performance of the SAP. This will allow the composite absorbent body containing the polymer absorbent to more stably exhibit absorption performance as an absorbent body.

The ion-exchange rate of the polymer absorbent for polyvalent ions is more preferably 60% or greater and even more preferably 70% or greater.

The ion-exchange rate of the polymer absorbent for polyvalent ions can be measured by any method such as ICP luminescence analysis, IC analysis, or atomic absorption spectrometry, and for example, the ion-exchange rate for divalent ions may be measured in the following manner.

### <Measurement of Ion-Exchange Rate of Polymer Absorbent for Divalent Ions>

(1) Artificial urine is prepared by dissolving 200 g of urea, 80 g of sodium chloride, 8 g of magnesium sulfate, 3 g of calcium chloride and approximately 1 g of dye (Blue #1) in 10 L of ion-exchanged water.
(2) The divalent ion concentration (mEq/L) of the prepared artificial urine is measured using an ion meter (HORIBA LAQUAtwin-Ca-11 compact calcium ion meter by Horiba Advanced Techno Co., Ltd.). The measured ion concentration is recorded as the “ion concentration before polymer absorbent contact”.
(3) A 0.2 g portion of the polymer absorbent measuring sample is placed in a glass filter (climbing glass filter, Model: 0777-01-101, outer diameter×foot length (mm):φ7×80, filter diameter: φ20 mm, volume: 30 mL, material: borosilicate glass, pore size: 100 to 120 μm), and 30 mL of the artificial urine is poured in, using the ion meter to measure the divalent ion concentration (mEq/L) of the resulting filtrate. The measured ion concentration is recorded as the “ion concentration after polymer absorbent contact”.
(4) The ion concentration after polymer absorbent contact is subtracted from the ion concentration before polymer absorbent contact to calculate the change in ion concentration (mEq/L) before and after polymer absorbent contact, and the change in ion concentration (mEq/L) before and after polymer absorbent contact is also divided by the ion concentration before polymer absorbent contact and multiplied by 100 to calculate the rate of change (%) in the divalent ion concentration. As used herein, “rate of change (%) in the divalent ion concentration” is the “divalent ion exchange rate of the polymer absorbent”.

The measurement is carried out under conditions with a temperature of 25° C. and a humidity of 60%.

When the measuring sample (polymer absorbent) used is to be collected from a product, it can be obtained by the following <Measuring sample (polymer absorbent) collection method>.

<Measuring sample (polymer absorbent) collection method>
(1) The front sheet is peeled from the product to expose the absorbent body.
(2) The material to be measured (polymer absorbent) is dropped out from the exposed absorbent body and materials other than the (particulate) material to be measured (for example, pulp and synthetic resin fibers) are removed using forceps.
(3) A microscope or simple magnifying lens is used as magnified observation means for observation at a magnification allowing discrimination of SAP differences or a magnification allowing visualization of pores in the porous body, while using the forceps to collect the material to be measured. The magnification of the simple magnifying lens is not particularly restricted so long as it is a magnification allowing visualization of the pores in the porous body, and it may be 25× or 50× magnification, for example.
(4) The collected material to be measured is used as a sample for measurement according to different measuring methods.

According to the invention the polymer absorbent preferably has a liquid absorption of 30 g/g or greater per unit mass. If the liquid absorption of the polymer absorbent is at least a certain value, it will be possible to reliably absorb and modify more liquid, helping to reduce variation in the absorption performance of the SAP. This will allow the composite absorbent body containing the polymer absorbent to more stably exhibit absorption performance as an absorbent body.

The liquid absorption of the polymer absorbent per unit mass is more preferably 40 g/g or greater and even more preferably 50 g/g or greater.

The liquid absorption of the polymer absorbent per unit mass can be measured in the following manner.

### <Measurement of Liquid Absorption of Polymer Absorbent Per Unit Mass>

(1) A 1 g portion of sample to be measured (polymer absorbent) is encapsulated in a mesh bag cut to 10 cm square (N-NO255HD 115 by NBC Meshtec, Inc. (standard width: 115 cm, 255 mesh/2.54 cm, opening: 57 lam, filament diameter: 43 lam, thickness: 75 lam). The mass (g) of the mesh bag is measured in advance. When the measuring sample (polymer absorbent) used is to be collected from a product, it can be obtained according to <Measuring sample (polymer absorbent) collection method> above.
(2) The sample-encapsulating mesh bag is immersed in a 0.9% aqueous sodium chloride solution for 1 hour.
(3) The mass (g) of the mesh bag is measured after hang draining for 5 minutes.
(4) The total of the mass of the sample (=1 g) and the mesh bag is subtracted from the mass of the drained mesh bag measured in (3) to calculate the liquid absorption (g) of the sample, and the liquid absorption is then divided by the mass of the sample (=1 g) to obtain the liquid absorption per unit mass (g/g) for the sample (polymer absorbent).

The measurement is carried out under conditions with a temperature of 25° C. and a humidity of 60%.

The method for producing the polymer absorbent will now be described in detail for the aforementioned absorbent A as an example.

### [Method for Producing Polymer Absorbent]

As shown in FIG. 3, absorbent A can be obtained by a crosslinking polymerization step and a hydrolysis step. The steps will now be explained in detail.

### (Crosslinking Polymerization Step)

First, an oil-soluble monomer for crosslinking polymerization, a crosslinkable monomer, a surfactant and water, with a polymerization initiator as necessary, are mixed to obtain a water-in-oil emulsion. The water-in-oil emulsion is an emulsion having an oil phase as the continuous phase, containing dispersed water droplets.

For absorbent A, as shown at top of FIG. 3, crosslinking polymerization is carried out using the (meth)acrylic acid ester butyl methacrylate as the oil-soluble monomer, divinylbenzene as the crosslinkable monomer, sorbitan monooleate as the surfactant and azobis(isobutyronitrile) as the polymerization initiator, to obtain monolith A.

Specifically, for absorbent A, as indicated at top of FIG. 3, 9.2 g of t-butyl methacrylate as the oil-soluble monomer, 0.28 g of divinylbenzene as the crosslinkable monomer, 1.0 g of sorbitan monooleate (SMO) as the surfactant and 0.4 g of 2,2′-azobis(isobutyronitrile) as the polymerization initiator are mixed and homogeneously dissolved.

The mixture of t-butyl methacrylate/divinylbenzene/SMO/2,2′-azobis(isobutyronitrile) is then added to 180 g of purified water, and the resulting mixture is stirred under reduced pressure using a vacuum stirring/degassing mixer (product of EMI) as a planetary stirrer, to obtain a water-in-oil emulsion.

The emulsion is rapidly transferred to a reactor and sealed, and then polymerization is carried out under stationary conditions of 60° C., 24 hours. Upon completion of polymerization, the contents are removed out, extracted with methanol, and dried under reduced pressure, to obtain monolith A having a continuous macropore structure. Observation of the internal structure of monolith A by SEM showed that monolith A had an open-cell structure and a continuous skeleton thickness of 5.4 μm. The mean diameter of the continuous pores measured by the mercury intrusion method was 36.2 μm, and the total pore volume was 15.5 mL/g.

The divinylbenzene content with respect to the total monomer is preferably 0.3 to 10 mol % and more preferably 0.3 to 5 mol %. The proportion of divinylbenzene with respect to the total of butyl methacrylate and divinylbenzene is preferably 0.1 to 10 mol % and more preferably 0.3 to 8 mol %. For absorbent A, the proportion of butyl methacrylate with respect to the total of butyl methacrylate and divinylbenzene is 97.0 mol %, and the proportion of divinylbenzene is 3.0 mol %.

The amount of surfactant added can be set according to the type of oil-soluble monomer and the desired size of the emulsion particles (macropores), being preferably in the range of about 2 to 70% with respect to the total amount of the oil-soluble monomer and surfactant.

For control of the foamed form and size of monolith A, an alcohol such as methanol or stearyl alcohol; a carboxylic acid such as stearic acid; a hydrocarbon such as octane, dodecane or toluene; or a cyclic ether such as tetrahydrofuran or dioxane may be added into the polymerization system.

The mixing method for formation of the water-in-oil emulsion is not particularly restricted, and for example, it may be a method of mixing each of the components all at once, or a method of mixing each of the components after having separately and homogeneously dissolved the oil-soluble monomer, surfactant and oil-soluble polymerization initiator as the oil-soluble components, and the water and water-soluble polymerization initiator, as the water-soluble components.

The mixer used to form the emulsion is also not particularly restricted, and any apparatus such as an ordinary mixer, homogenizer or high-pressure homogenizer may be employed, depending on the desired particle size for the emulsion, or a planetary stirrer may be used, wherein the material to be treated is placed in a mixing vessel and the material is stirred and mixed by rotation of the mixing vessel while it revolves around a revolution axis in an inclined state.

The mixing conditions are also not particularly restricted, and the stirring rotational speed and stirring time may be arbitrarily set, depending on the desired particle size for the emulsion. A planetary stirrer as described above can form homogeneous water droplets in a W/O emulsion, and the mean diameter may be set within a wide range.

The polymerization conditions for the water-in-oil emulsion may be any of various conditions set depending on the type of monomer or initiator. For example, when using azobis(isobutyronitrile), or benzoyl peroxide or potassium persulfate as the polymerization initiator, thermal polymerization may be carried out in a sealed vessel under an inert atmosphere at a temperature of 30 to 100° C. for 1 to 48 hours, or when using hydrogen peroxide-ferrous chloride or sodium persulfate-acidic sodium sulfite as the polymerization initiator, polymerization may be carried out in a sealed vessel under an inert atmosphere at a temperature of 0 to 30° C. for 1 to 48 hours.

Upon completion of polymerization, the contents may be removed and subjected to Soxhlet extraction with a solvent such as isopropanol to remove the unreacted monomer and residual surfactant, to obtain monolith A shown at center in FIG. 3.

### (Hydrolysis Step)

A subsequent step of hydrolysis of monolith A (crosslinked polymer) to obtain absorbent A (hydrolysis step) will now be explained.

First, monolith A is immersed in zinc bromide-added dichloroethane and stirred at 40° C. for 24 hours, contacted with methanol, 4% hydrochloric acid, 4% aqueous sodium hydroxide and water in that order for hydrolysis, and then dried to obtain absorbent A in block form. The block absorbent A is pulverized to the desired size to obtain particulate absorbent A. The form of absorbent A is not limited to being particulate, and may, for example, be formed into a sheet either during or after drying.

The hydrolysis method for monolith A is not particularly restricted, and any of various methods may be applied. For example, it may be a method of contacting an aromatic solvent such as toluene or xylene, a halogen-based solvent such as chloroform or dichloroethane, an ether-based solvent such as tetrahydrofuran or isopropyl ether, an amide-based solvent such as dimethylformamide or dimethyl acetamide, an alcohol-based solvent such as methanol or ethanol, or a carboxylic acid-based solvent such as acetic acid or propionic acid, or water, as a solvent, with a strong base such as sodium hydroxide, or a method of contacting with a hydrohalic acid such as hydrochloric acid, a Bronsted acid such as sulfuric acid, nitric acid, trifluoroacetic acid, methanesulfonic acid or p-toluenesulfonic acid, or a Lewis acid such as zinc bromide, aluminum chloride, aluminum bromide, titanium(IV) chloride, cerium chloride/sodium iodide or magnesium iodide.

The (meth)acrylic acid ester in the polymerization material for the organic polymer that is to form the hydrophilic continuous skeleton of absorbent A is not particularly restricted; however, it is preferably a C1-C10 (1 to 10 carbon atom) alkyl ester of (meth)acrylic acid, and most preferably a C4 (4 carbon atom) alkyl ester of (meth)acrylic acid.

C4 alkyl esters of (meth)acrylic acid include t-butyl (meth)acrylate esters, n-butyl (meth)acrylate esters and iso-butyl (meth)acrylate esters.

The monomers to be used for crosslinking polymerization may be a (meth)acrylic acid ester and divinylbenzene alone, or they may include monomers other than a (meth)acrylic acid ester and divinylbenzene, in addition to the (meth)acrylic acid ester and divinylbenzene.

In the latter case, the other monomers are not particularly restricted; however, include, for example, styrene, α-methylstyrene, vinyltoluene, vinylbenzyl chloride, glycidyl (meth)acrylate, 2-ethylhexyl (meth)acrylate, isobutene, butadiene, isoprene, chloroprene, vinyl chloride, vinyl bromide, vinylidene chloride, tetrafluoroethylene, (meth)acrylonitrile, vinyl acetate, ethylene glycol di(meth)acrylate and trimethylolpropane tri(meth)acrylate.

The proportion of monomers other than the (meth)acrylic acid ester and divinylbenzene in the total monomers used for crosslinking polymerization is preferably 0 to 80 mol % and more preferably 0 to 50 mol %.

The surfactant is not limited to sorbitan monooleate mentioned above and may be any one that can form a water-in-oil (W/O) emulsion when mixed with the crosslinking polymerization monomer and water. Examples of surfactants include nonionic surfactants such as sorbitan monolaurate, sorbitan monopalmitate, sorbitan monostearate, sorbitan trioleate, polyoxyethylene nonylphenyl ether, polyoxyethylene stearyl ether and polyoxyethylene sorbitan monooleate, anionic surfactants such as potassium oleate, sodium dodecylbenzenesulfonate and dioctylsodium sulfosuccinate, cationic surfactants such as distearyldimethylammonium chloride, and amphoteric surfactants such as lauryldimethyl betaine. Such surfactants may be used alone, or two or more may be used in combination.

The polymerization initiator used may be a compound that generates radicals under heat or photoirradiation. The polymerization initiator may be water-soluble or oil-soluble, and examples include azobis(4-methoxy-2,4-dimethylvaleronitrile), azobis(isobutyronitrile), azobisdimethylvaleronitrile, azobiscyclohexanenitrile, azobiscyclohexanecarbonitrile, azobis(2-methylpropionamidine) dihydrochloride, benzoyl peroxide, potassium persulfate, ammonium persulfate, hydrogen peroxide-ferrous chloride, sodium persulfate-acidic sodium sulfite and tetramethylthiuram disulfide. Depending on the system, however, polymerization may proceed by heating alone or photoirradiation alone without addition of a polymerization initiator, and in such cases there is no need to add a polymerization initiator.

The composite absorbent body of the invention is not particularly restricted and can be applied as a composite absorbent body for a wide range of fields including civil engineering and construction materials such as condensation-proof sheets or simple soil, base materials for pharmaceuticals and the like, and absorption materials for leaking liquids. The liquids to be absorbed by the composite absorbent body are also not particularly restricted, and examples include water and water-soluble solutions (such as seawater), acids (such as hydrochloric acid), bases (such as sodium hydroxide), and organic solvents (including alcohols such as methanol and ethanol, ketones such as acetone, ethers such as tetrahydrofuran (THF) and 1,4-dioxane, N,N-dimethylformamide (DMF) and dimethyl sulfoxide (DMSO)). Such liquids may also be mixtures of two or more liquids.

The invention is also not restricted to the embodiments described above and can incorporate appropriate combinations, substitutions and modifications within a range that is not outside of the object and gist of the invention. Incidentally, the ordinal terms “first” and “second” as used throughout the present specification serve merely to distinguish between the numbered embodiments, and are not used to indicate any relative ordering, precedence or importance.

### REFERENCE SIGNS LIST

1 Composite absorbent body
2 First retaining sheet
3 Second retaining sheet
4 Polymer absorbent
5 Superabsorbent polymer (SAP)
6 Hydrophilic fiber sheet

1. A composite absorbent body for absorption of liquid, wherein:
the composite absorbent body includes a polymer absorbent comprising a hydrophilic continuous skeleton and continuous pore, and a superabsorbent polymer, and
the polymer absorbent contains at least a —COOH group and a —COONa group as ion-exchange groups, a total ion-exchange capacity for —COOH and —COONa groups being 4.0 mg equivalents/g or greater per mass in a dry state.

2. The composite absorbent body according to claim 1, wherein the polymer absorbent has an ion-exchange rate of 50% or greater for a polyvalent ion.

3. The composite absorbent body according to claim 1, wherein the polymer absorbent has a liquid absorption of 30 g/g or greater per unit mass.

4. The composite absorbent body according to claim 1, wherein the polymer absorbent has a void percentage of 85% or greater per unit volume of the polymer absorbent.

5. The composite absorbent body according to claim 1, wherein the polymer absorbent has a mean diameter of 1 μm to 1000 μm for the continuous pores.

6. The composite absorbent body according to claim 1, wherein the polymer absorbent is a monolithic absorbent.

7. The composite absorbent body according to claim 1, wherein the polymer absorbent is a crosslinked polymer hydrolysate of a compound comprising a (meth)acrylic acid ester and two or more vinyl groups in a molecule.

8. The composite absorbent body according to claim 1, wherein the superabsorbent polymer is an acrylic acid-based superabsorbent polymer with a cation on a surface.

9. A polymer absorbent to be used with a superabsorbent polymer, wherein:
the polymer absorbent comprises a hydrophilic continuous skeleton and continuous pore, and
contains at least a —COOH group and a —COONa group as ion-exchange groups, a total ion-exchange capacity for —COOH and —COONa groups being 4.0 mg equivalents/g or greater per mass in a dry state.
